(19) United States
(12) Patent Application Publication
(10) Pub. No.: US 2026/0207577 A1
(43) Pub. Date: Jul. 23, 2026

(54) METHODS OF USING FACTOR B INHIBITORS

(71) Applicant: Novartis AG, Basel (CH)

(72) Inventors: Matthias MEIER, Grenzach-Wyhlen (DE); Udaykiran VELDANDI, Hyperabad (IN); Yaqin WANG, Sandy, UT (US); Nicholas WEBB, Basel (CH)

(21) Appl. No.: 19/158,142

(22) PCT Filed: Feb. 22, 2024
(86) PCT No.: PCT/IB2024/051729

§ 371 Date: Aug. 20, 2025

(30) Foreign Application Priority Data
Feb. 23, 2023 (IN) 202311012279

(51) Int. Cl. A61K 31/454; A61K 45/06; A61P 13/12; A61P 37/06

(52) U.S. Cl. CPC A61K 31/454; A61K 45/06; A61P 13/12; A61P 37/06

(57) ABSTRACT

Described herein are methods of treating immune complex mediated membranoproliferative glomerulonephritis (IC-MPGN) with the Factor B inhibitor iptacopan or a pharmaceutically acceptable salt thereof, e.g. iptacopan hydrochloride.

### FIELD

The disclosure relates to methods of treating complement mediated diseases, and in particular, immune complex-mediated membranoproliferative glomerulonephritis (IC-MPGN) with the Factor B inhibitor iptacopan or a pharmaceutically acceptable salt thereof, e.g. iptacopan hydrochloride.

### BACKGROUND

Immune-complex membranoproliferative glomerulonephritis (IC-MPGN) is an ultra-rare, fast-progressing kidney disease, that can be idiopathic (primary IC-MPGN) or may be due to secondary to chronic infections or other immunological systemic diseases (Sethi S, Fervenza FC (2011) Membranoproliferative glomerulonephritis: pathogenetic heterogeneity and proposal for a new classification. Semin Nephrol; 31(4):341-8). Although IC-MPGN is a separate disease as per current classification, it has key similarities to C3 glomerulopathy (C3G), which is also typically characterized by membranoproliferative histopathology. IC-MPGN has predominant immunoglobulin complex staining, while C3G has predominant complement-3 staining.

Both IC-MPGN and C3G are ultra-rare (Wong E K S, Marchbank K J, Lomax-Browne H, et al (2021) C3 Glomerulopathy and Related Disorders in Children: Etiology-Phenotype Correlation and Outcomes. Clin J Am Soc Nephrol; 16(11):1639-51), both are frequently diagnosed in childhood and adolescence, with a median age at diagnosis of around 21 years for IC-MPGN (Iatropoulos P, Daina E, Curreri M, et al (2018) Cluster Analysis Identifies Distinct Pathogenetic Patterns in C3 Glomerulopathies/Immune Complex-Mediated Membranoproliferative GN. J Am Soc Nephrol; 29(1):283-94; and Garam N, Prohnszka Z, Szilágyi Á, et al (2020) Validation of distinct pathogenic patterns in a cohort of membranoproliferative glomerulonephritis patients by cluster analysis. Clin Kidney J; 13(2):225-34) and both are characterized by a high risk of progression to kidney failure (Iatropoulos et al 2018; and Wong et al 2021). Dysregulation of the alternative pathway (AP) of the complement system is strongly implicated in the pathogenesis of IC-MPGN as well as in C3G (Wong et al 2021).

Membranoproliferative glomerulonephritis (MPGN) is a rare histologic pattern of glomerular injury characterized on renal biopsy by the findings of hypercellularity, endocapillary proliferation and capillary wall thickening with double contour formation at light microscopy level. It has many causes, and its detection should prompt the clinician to search for the potential underlying etiology. Recent improvements in the understanding of the pathogenesis, with the recognition of the pivotal role of the alternative complement pathway (AP), have led to the description of complement-mediated (C3 glomerulopathy, C3G) and immune complex-mediated (IC-MPGN) forms (Schena F P, Esposito P, Rossini M (2020) A Narrative Review on C3 Glomerulopathy: A Rare Renal Disease. Int J Mol Sci; 21(2):525). A diagnosis of C3G is made where there is dominant glomerular C3 deposition and little or no Ig deposition (C3 greater than or equal to two grades of order of magnitude greater than any other immune reactant), while IC-MPGN is diagnosed when there is mostly glomerular Ig deposition (Sethi, Fervenza 2011; Cook H T, Pickering M C (2015) Histopathology of MPGN and C3 glomerulopathies. Nat Rev Nephrol; 11(1):14-22) based on the findings from immunofluorescence staining of the kidney biopsy. There is evidence that histological diagnosis may change with time; among patients who underwent repeat biopsies, 40% have different immunofluorescence (IF) staining patterns on the initial and follow-up biopsies and 17% exhibited a shift from C3G to IC-MPGN and vice versa (Hou J, Markowitz G S, Bomback A S, et al (2014) Toward a working definition of C3 glomerulopathy by immunofluorescence. Kidney Int; 85(2):450-6). C3G is further classified into C3 glomerulonephritis (C3GN) and dense deposit disease (DDD) according to additional findings seen at electron microscopy level only.

IC-MPGN may develop secondary to chronic infections including hepatitis B or C virus infection, endocarditis, shunt nephritis, abscesses, fungal infections, parasitic infections such as schistosomiasis, echinococcosis, malaria, etc.), or systemic autoimmune disorders including but not exclusively SLE, Sjogren syndrome and rheumatoid arthritis and last but not least monoclonal gammopathies. As such a careful evaluation for underlying causes is essential. Treatment of such secondary cases is generally directed at the underlying condition. Where no such underlying cause is detected, the IC-MPGN is classified as idiopathic (primary IC-MPGN). Here a complete complement workup should be performed as data support a key role for complement dysregulation in IC-MPGN. This work-up should include functional complement assays, quantification of complement components and regulators, measurement of complement activation, complement autoantibodies, genetic testing, and immunofluorescent staining of kidney biopsy samples (KDIGO 2021). Several studies indicate approximately equal numbers of primary IC-MPGN and C3G patients (Servais A, Noël LH, Roumenina L T, et al (2012) Acquired and genetic complement abnormalities play a critical role in dense deposit disease and other C3 glomerulopathies. Kidney Int; 82(4):454-64; Iatropoulos et al 2016, Wong et al 2021) pointing towards an incidence of 1-2 per million per year (Medjeral-Thomas et al 2014).

The clinical presentation of IC-MPGN is heterogeneous and ranges from asymptomatic hematuria and proteinuria, nephritic or nephrotic syndrome or rapidly progressive glomerulonephritis with acute kidney injury (Iatropoulos et al 2016; Holle J, Berenberg-Goßler L, Wu K, et al (2018) Outcome of membranoproliferative glomerulonephritis and C3-glomerulopathy in children and adolescents. Pediatr Nephrol; 33(12):2289-98). MPGN pattern may exist without Ig deposits but not without C3 deposits. In serial kidney biopsies in each patient, Ig deposits are variable and may disappear and C3 deposits are constantly present. Nephrotic syndrome is more frequent in IC-MPGN (43%-70%) compared to C3G (26%-52%) patients. Glomerular C4d staining is positive in a majority (80%) of primary (limited number of studied cases) and secondary Ig-MPGN and only (and faintly) in a minority (13%) of C3G cases. IC-MPGN is a rapidly progressing disease, with an estimated 50% of patients progressing to kidney failure within 10 years (Servais et al 2012). This is similar to what was found for C3G patients in this study, although some recent data suggests progression to kidney failure in IC-MPGN may be somewhat less rapid than in C3G (Lomax-Browne H J, Medjeral-Thomas N R, Barbour S J, et al (2022) Association of Histologic Parameters with Outcome in C3 Glomerulopathy and Idiopathic Immunoglobulin-Associated Membranoproliferative Glomerulonephritis. Clin J Am Soc Nephrol; 17(7):994-1007).

Supportive therapy with renin angiotensin system inhibition alone is recommended for patients with idiopathic IC-MPGN and proteinuria<3.5 g/d and a normal estimated glomerular filtration rate (eGFR). Similar therapy is also recommended for those patients who present late when active disease has subsided and there is advanced tubulointerstitial fibrosis on kidney biopsy.

The Clinical Practice Guideline for Glomerulonephritis (KDIGO 2021) recognize that there is currently no evidence to support the use of immunosuppressive therapy in adults or children with primary IC-MPGN. However for patients with nephrotic syndrome and normal or near normal GFR, a limited course of corticosteroids is recommended, or where a contraindication to corticosteroids exists, a calcineurin inhibitor may be used. The guidelines note that immunosuppressive therapy may be commenced at lower levels of proteinuria in children, who may additionally be more likely to receive MMF as a corticosteroid sparing agent. For patients with abnormal kidney function, glucocorticoids and immunosuppressive therapy should be added to supportive care (KDIGO 2021).

The complement alternative pathway (AP) is important for innate and adaptive immunity. However, hyperactivity of AP is known to cause and worsen a wide number of diseases with autoimmune components.

Iptacopan is a first-in-class, oral, low molecular weight (LMW) reversible and selective inhibitor of Factor B (FB) (Schubart et al 2019, Small-molecule factor B inhibitor for the treatment of complement-mediated diseases. Proc Natl Acad Sci USA p. 7926-7931), a key protease of the AP (Merle et al 2015, Complement System Part I—Molecular Mechanisms of Activation and Regulation. Front Immunol p. 262). Inhibition of FB prevents amplification of all pathways as well as AP-induced assembly of C3- and C5-convertases. At the same time, iptacopan has only limited effect on classical-pathway induced activation of the terminal pathway. Iptacopan inhibits FB in the context of the C3 convertase and thereby blocks AP-dependent C3 activation and the amplification of CP- and LP-dependent C5 activation. Iptacopan does not, however, block the generation of MAC initiated by CP and LP. This is important, since it means that in immunized individuals, MAC-dependent killing of Neisseria species through activation of CP will be maintained. Nevertheless, no clinical data is available using iptacopan in IC-MPGN patients.

In summary, the mechanism and underlying cause of the formation of glomerular immune-complexes in IC-MPGN is unknown. Additionally, although the clinical presentation and disease course (including rate of progression to kidney failure) in IC-MPGN are similar to those in C3G, the occurrence of nephrotic syndrome is more frequent (43%-70%) in IC-MPGN patients compared to C3G (26%-52%) patients. No clinical data is yet available using complement inhibitors in IC-MPGN patients.

### SUMMARY

The disclosure relates to methods of treating complement mediated diseases, and in particular, immune complex-mediated membranoproliferative glomerulonephritis (IC-MPGN) with iptacopan (Formula I, shown below) or a pharmaceutically acceptable salt thereof, e.g. iptacopan hydrochloride. Iptacopan is also known as LNP023. The terms “iptacopan” and “LNP023” are used herein interchangeably. Iptacopan (4-((2S,4S)-(4-ethoxy-1-((5-methoxy-7-methyl-1H-indol-4-yl)methyl)piperidin-2-yl))benzoic acid) belongs to the class of Factor B inhibitors of the complement pathway and acts by inhibiting or suppressing the amplification of the complement system caused by C3 activation irrespective of the initial mechanism of activation.

Iptacopan hydrochloride is chemically designated as 4-((2S,4S)-(4-ethoxy-1-((5-methoxy-7-methyl-1H-indol-4-yl)methyl)piperidin-2-yl))benzoic acid hydrochloride as shown in Formula I.

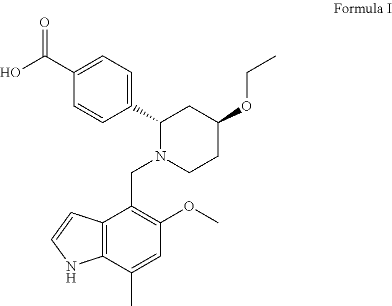 [SMILES: CCO[C@H]1CCN(Cc2c(OC)cc(C)c3[nH]ccc23)[C@H](c2ccc(C(=O)O)cc2)C1]

Iptacopan hydrochloride and methods for its preparation are disclosed in WO2015/009616 (see Example 26d), which is incorporated herein by reference in its entirety. The form of iptacopan hydrochloride used as the investigational study drug for this study is a monohydrate (Form HB) as shown in the formula below:

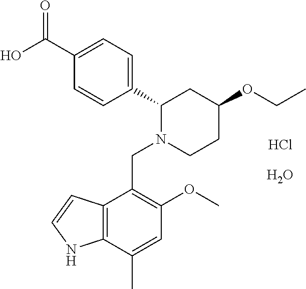 [SMILES: CCO[C@H]1CCN(Cc2c(OC)cc(C)c3[nH]ccc23)[C@H](c2ccc(C(=O)O)cc2)C1 ; O ; Cl]

### (2S,4S)-2-(4-Carboxyphenyl)-4-ethoxy-1-[(5-methoxy-7-methyl-1H-indol-4-yl)methyl]piperidin-1-ium chloride-water (1/1)

Iptacopan hydrochloride monohydrate Form HB and methods for its preparation are disclosed in U.S. Ser. No. 63/026,637 and U.S. Ser. No. 63/052,699 published in WO 2021/234544, each of which is incorporated herein by reference in its entirety.

The disclosure provides a method of treating immune complex-mediated membranoproliferative glomerulonephritis (IC-MPGN) in a subject, e.g., a patient, in need thereof, the method comprising administering, e.g., orally, to the subject, e.g., patient, iptacopan or a pharmaceutically acceptable salt thereof, e.g., iptacopan hydrochloride, e.g., at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours, to thereby treat the subject, e.g., patient (wherein the dosing amount refers to the anhydrous free base of iptacopan hydrochloride), to thereby treat the subject, e.g., a patient.

In one aspect, the present invention provides a method of treating immune complex mediated membranoproliferative glomerulonephritis (IC-MPGN) in a subject in need thereof, the method comprising administering to the subject iptacopan or a pharmaceutically acceptable salt thereof to thereby treat the subject.

In a further aspect, the present invention provides iptacopan or a pharmaceutically acceptable salt thereof for use in a treatment of immune complex mediated membranoproliferative glomerulonephritis (IC-MPGN) in a subject in need thereof, wherein the treatment comprises administering to the subject iptacopan or a pharmaceutically acceptable salt thereof.

In another aspect, the present invention provides a pharmaceutical composition comprising a therapeutically effective amount of iptacopan or a pharmaceutically acceptable salt thereof for use in treating immune complex mediated membranoproliferative glomerulonephritis (IC-MPGN), in a subject in need thereof, wherein the pharmaceutical composition is to be administered to thereby treat the subject.

In another aspect, the present invention provides use of iptacopan or a pharmaceutically acceptable salt thereof for the manufacture for treating immune complex mediated membranoproliferative glomerulonephritis (IC-MPGN), in a subject in need thereof.

In one embodiment, the methods and uses of the present invention comprise administering iptacopan or a pharmaceutically acceptable salt thereof to the subject at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, administered twice daily (b.i.d.), wherein the dosing amount refers to the anhydrous free base of iptacopan. In one embodiment, the methods and uses of the present invention comprise administering iptacopan or a pharmaceutically acceptable salt thereof to the subject at a dose of 200 mg, administered twice daily (b.i.d.), wherein the dosing amount refers to the anhydrous free base of iptacopan.

Non-limiting embodiments of the present disclosure are described in the following embodiments:
Embodiment 1: A method of treating immune complex mediated membranoproliferative glomerulonephritis (IC-MPGN) in a subject in need thereof, the method comprising administering to the subject iptacopan or a pharmaceutically acceptable salt thereof to thereby treat the subject.
Embodiment 2: The method of embodiment 1, wherein the iptacopan or a pharmaceutically acceptable salt thereof is administered orally.
Embodiment 3: The method of embodiment 1 or 2, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, administered twice daily (b.i.d.), wherein the dosing amount refers to the anhydrous free base of iptacopan.
Embodiment 4: The method of embodiment 3, wherein the dose is about 200 mg, twice daily.
Embodiment 5: The method of any one of embodiments 1 to 4, wherein the pharmaceutically acceptable salt of iptacopan is iptacopan hydrochloride.
Embodiment 6: The method of any of embodiments 1 to 5, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.
Embodiment 7: The method of embodiment 6, wherein the ACEi or ARB has been administered at a maximally recommended or tolerated dose.
Embodiment 8: The method of embodiment 7, wherein the subject is further administered the supportive care at a stable dose.
Embodiment 9: The method of any of embodiments 1 to 8, wherein the subject is an adult or adolescent.
Embodiment 10: The method of any of embodiments 1 to 9, wherein a urine protein/creatinine ratio (UPCR) in the subject is reduced compared to prior to administering iptacopan or a pharmaceutically acceptable salt thereof.
Embodiment 11: The method of embodiment 10, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.
Embodiment 12: The method of any of embodiments 1 to 11, wherein an estimated glomerular filtration rate (eGFR) in the subject is stable or improved, compared to prior to administering iptacopan or a pharmaceutically acceptable salt thereof.
Embodiment 13: The method of embodiment 12, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.
Embodiment 14: Iptacopan or a pharmaceutically acceptable salt thereof for use in a treatment of immune complex mediated membranoproliferative glomerulonephritis (IC-MPGN) in a subject in need thereof, wherein the treatment comprises administering to the subject iptacopan or a pharmaceutically acceptable salt thereof.
Embodiment 15: The iptacopan or a pharmaceutically acceptable salt thereof for use of embodiment 14, wherein the iptacopan or a pharmaceutically acceptable salt thereof is administered orally.
Embodiment 16: The iptacopan or a pharmaceutically acceptable salt thereof for use of embodiment 14 or 15, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), wherein the dosing amount refers to the anhydrous free base of iptacopan.
Embodiment 17: The iptacopan or a pharmaceutically acceptable salt thereof for use of embodiment 16, wherein the dose is about 200 mg, twice daily.
Embodiment 18: The iptacopan or a pharmaceutically acceptable salt thereof for use of any one of embodiments 14 to 17, wherein the pharmaceutically acceptable salt of iptacopan is iptacopan hydrochloride.
Embodiment 19: The iptacopan or a pharmaceutically acceptable salt thereof for use of any of embodiments 14 to 18, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.
Embodiment 20: The iptacopan or a pharmaceutically acceptable salt thereof for use of embodiment 19, wherein the ACEi or ARB has been administered at a maximally recommended or tolerated dose.
Embodiment 21: The iptacopan or a pharmaceutically acceptable salt thereof for use of embodiment 20, wherein the subject is further administered the supportive care at a stable dose.
Embodiment 22: The iptacopan or a pharmaceutically acceptable salt thereof for use of any of embodiments 14 to 21, wherein the subject is an adult or adolescent.
Embodiment 23: The iptacopan or a pharmaceutically acceptable salt thereof for use of any of embodiments 14 to 22, wherein a urine protein/creatinine ratio (UPCR) in the subject is reduced compared to prior to administering iptacopan or a pharmaceutically acceptable salt thereof.
Embodiment 24: The iptacopan or a pharmaceutically acceptable salt thereof for use of embodiment 23, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.
Embodiment 25: The iptacopan or a pharmaceutically acceptable salt thereof for use of embodiments 14 to 24, wherein an estimated glomerular filtration rate (eGFR) in the subject is stable or improved, compared to prior to administering iptacopan or a pharmaceutically acceptable salt thereof.
Embodiment 26: The iptacopan or a pharmaceutically acceptable salt thereof for use of embodiment 25, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.
Embodiment 27: A pharmaceutical composition comprising a therapeutically effective amount of iptacopan or a pharmaceutically acceptable salt thereof for use in treating immune complex mediated membranoproliferative glomerulonephritis (IC-MPGN), in a subject in need thereof, wherein the pharmaceutical composition is to be administered to thereby treat the subject.
Embodiment 28: The pharmaceutical composition of embodiment 27, wherein the iptacopan or a pharmaceutically acceptable salt thereof is administered orally.
Embodiment 29: The pharmaceutical composition of embodiment 27 or 28, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), wherein the dosing amount refers to the anhydrous free base of iptacopan.
Embodiment 30: The pharmaceutical composition of embodiment 29, wherein the dose is about 200 mg, twice daily.
Embodiment 31: The pharmaceutical composition of any one of embodiments 27 to 30, wherein the pharmaceutically acceptable salt of iptacopan is iptacopan hydrochloride.
Embodiment 32: The pharmaceutical composition of any one of embodiments 27 to 31, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.
Embodiment 33: The pharmaceutical composition of embodiment 32, wherein the ACEi or ARB has been administered at a maximally recommended or tolerated dose.
Embodiment 34: The pharmaceutical composition of embodiment 33, wherein the subject is further administered the supportive care at a stable dose.
Embodiment 35: The pharmaceutical composition of any one of embodiments 27 to 34, wherein the subject is an adult or adolescent.
Embodiment 36: The pharmaceutical composition of any one of embodiments 27 to 35, wherein a urine protein/creatinine ratio (UPCR) in the subject is reduced compared to prior to administering iptacopan or a pharmaceutically acceptable salt thereof.
Embodiment 37: The pharmaceutical composition of embodiment 35, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.
Embodiment 38: The pharmaceutical composition of any one of embodiments 27 to 37, wherein an estimated glomerular filtration rate (eGFR) in the subject is stable or improved, compared to prior to administering iptacopan or a pharmaceutically acceptable salt thereof.
Embodiment 39: The pharmaceutical composition of embodiment 38, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.
Embodiment 40: Use of iptacopan or a pharmaceutically acceptable salt thereof for the manufacture for treating immune complex mediated membranoproliferative glomerulonephritis (IC-MPGN), in a subject in need thereof.
Embodiment 41: The use of embodiment 40, wherein the iptacopan or a pharmaceutically acceptable salt thereof is administered orally.
Embodiment 42: The use of embodiment 40 to 41, wherein the use comprises administering to the subjects iptacopan or a pharmaceutically acceptable salt thereof at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), wherein the dosing amount refers to the anhydrous free base of iptacopan.
Embodiment 43: The use of embodiment 42, wherein the dose is about 200 mg, twice daily.
Embodiment 44: The use of any one of embodiments 40 to 43, wherein the pharmaceutically acceptable salt of iptacopan is iptacopan hydrochloride.
Embodiment 45: The use of any one of embodiments 40 to 44, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.
Embodiment 46: The use of embodiment 45, wherein the ACEi or ARB has been administered at a maximally recommended or tolerated dose.
Embodiment 47: The use of embodiment 46, wherein the subject is further administered the supportive care at a stable dose.
Embodiment 48: The use of any one of embodiments 40 to 47, wherein the subject is an adult or adolescent.
Embodiment 49: The use of any one of embodiments 40 to 48, wherein a urine protein/creatinine ratio (UPCR) in the subject is reduced compared to prior to administering iptacopan or a pharmaceutically acceptable salt thereof.
Embodiment 50: The use of embodiment 49, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.
Embodiment 51: The use of any one of embodiments 40 to 50, wherein an estimated glomerular filtration rate (eGFR) in the subject is stable or improved, compared to prior to administering iptacopan or a pharmaceutically acceptable salt thereof.
Embodiment 52: The use of embodiment 51, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.

### BRIEF DESCRIPTION OF THE DRAWINGS

FIG. 1 depicts a schematic of the study design.

### DETAILED DESCRIPTION

Described herein is a method of treating immune complex mediated membranoproliferative glomerulonephritis IC-MPGN in a patient in need thereof using iptacopan or a pharmaceutically acceptable salt thereof, e.g., iptacopan hydrochloride, e.g., iptacopan hydrochloride monohydrate Form HB. Accordingly, described herein are methods of treating IC-MPGN in a patient in need thereof, the method comprising administering, e.g., orally, e.g., in capsule form, to the patient iptacopan or a pharmaceutically acceptable salt thereof, e.g., iptacopan hydrochloride, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg (wherein the dosing amount refers to the anhydrous free base of iptacopan hydrochloride), twice daily, e.g., about every 12 hours. Accordingly, described herein are methods of treating IC-MPGN in a patient in need thereof, the method comprising administering, e.g., orally, e.g., in capsule form, to the patient iptacopan or a pharmaceutically acceptable salt thereof, e.g., iptacopan hydrochloride, wherein the iptacopan or a pharmaceutically acceptable salt thereof is administered at a dose of 200 mg (wherein the dosing amount refers to the anhydrous free base of iptacopan hydrochloride), twice daily, e.g., about every 12 hours. Also described herein are methods of selecting the target patient population, methods of monitoring treatment of the target patient population, and methods of assessing safety and efficacy of treatment of the target patient population.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Remington's Pharmaceutical Sciences,” Mack Publishing Co., Easton, Pa., 21st edition, 2005, which is hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “about” means within ±10% of a value.

As used herein, “administering” or “administration” means providing a pharmaceutical agent to an individual, and includes, but is not limited to, administering by a medical professional and self-administering. Administration of a pharmaceutical agent to an individual can be continuous, chronic, short or intermittent.

As used herein, the term “acquire” or “acquiring” as the terms are used herein, refer to obtaining possession of a physical entity (e.g., a sample, e.g., a blood sample or a blood plasma sample), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy, e.g. LC-MS, e.g., LC-MS/MS methods.

As used herein, “dose” means a specified quantity of a pharmaceutical agent provided in a single administration, or in a specified time period. In certain embodiments, a dose can be administered in capsules. As used herein, the dosing amount refers to the anhydrous free base of iptacopan hydrochloride.

As used herein, “individual”, “patient”, “participant”, or “subject” means a human selected for treatment or therapy. As used herein, the term “adult” means an individual who is equal to or more than 18 years of age. As used herein, the term “adolescent” means an individual who is between 12 to 17 years of age.

As used herein, “pharmaceutically acceptable salts” means physiologically and pharmaceutically acceptable salts of iptacopan, i.e., salts that retain the desired biological activity of iptacopan and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of iptacopan may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Iptacopan hydrochloride and methods for its preparation are disclosed in WO2015/009616 (see Example 26d), which is incorporated herein by reference in its entirety.

As used herein, the term “treat”, “treating” or “treatment” means decrease, suppress, attenuate, diminish, arrest, or stabilize the development or progression of a disorder or disease, e.g., lupus nephritis, immune-complex membranoproliferative glomerulonephritis (IC-MPGN). Treatment does not require the complete curing of a disorder and encompasses the reduction of the symptoms or underlying risk factors or slowing a disease progression. As used herein, the terms “treat”, “treatment” and “treating” refer to the slowing, reduction or amelioration of the progression or severity of IC-MPGN or the amelioration of one or more symptoms, suitably of one or more discernible symptoms of IC-MPGN. In specific embodiments, the terms “treat”, “treatment” and “treating” refer to the amelioration of at least one measurable physical parameter of IC-MPGN (such as achieve or at least partially achieve a desired effect (e.g. proteinuria reduction, in particular as measured by Urine Protein:Creatinine Ratio (UPCR), improving estimated Glomerular Filtration Rate (eGFR), improvement in the Functional Assessment of Chronic Illness Therapy-Fatigue (FACIT-Fatigue) score), wherein the physical parameter is not necessarily discernible by the patient.

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

### METHODS OF USE

In one aspect, the disclosure provides a method of treating immune complex mediated membranoproliferative glomerulonephritis (IC-MPGN) in a subject, e.g., a patient, in need thereof, the method comprising administering, e.g., orally, to the subject, e.g., patient, iptacopan or a pharmaceutically acceptable salt thereof, e.g., iptacopan hydrochloride, e.g., iptacopan hydrochloride monohydrate Form HB, to thereby treat the subject, e.g., patient.

In another aspect, the disclosure provides a method of reducing proteinuria, e.g. reducing urine protein/creatinine ratio (UPCR), in a subject, e.g., a patient, in need thereof, the method comprising administering, e.g., orally, to the subject, e.g., patient, iptacopan or a pharmaceutically acceptable salt thereof, e.g., iptacopan hydrochloride, to thereby treat the subject, e.g., patient.

In another aspect, the disclosure provides a method of achieving a stable or improved estimated glomerular filtration rate (eGFR) in a subject, e.g., a patient, in need thereof, the method comprising administering, e.g., orally, to the subject, e.g., patient, iptacopan or a pharmaceutically acceptable salt thereof, e.g., iptacopan hydrochloride, e.g., iptacopan hydrochloride monohydrate Form HB, to thereby treat the subject, e.g., patient.

In another aspect, the disclosure provides iptacopan or a pharmaceutically acceptable salt thereof, e.g., iptacopan hydrochloride, e.g., iptacopan hydrochloride monohydrate Form HB, for use in the treatment of immune complex mediated membranoproliferative glomerulonephritis (IC-MPGN) in a subject, e.g., a patient, in need thereof, wherein the treatment comprises administering to the subject, e.g., patient, iptacopan or a pharmaceutically acceptable salt thereof, e.g., iptacopan hydrochloride, e.g., iptacopan hydrochloride monohydrate Form HB.

In another aspect, the disclosure provides iptacopan or a pharmaceutically acceptable salt thereof, e.g., iptacopan hydrochloride e.g., iptacopan hydrochloride monohydrate Form H_{B}, for use in reducing proteinuria, e.g., reducing UPCR, in a subject, e.g., a patient, in need thereof, wherein the treatment comprises administering to the subject, e.g., patient, iptacopan or a pharmaceutically acceptable salt thereof, e.g., iptacopan hydrochloride, e.g., iptacopan hydrochloride monohydrate Form HB.

In another aspect, the disclosure provides iptacopan or a pharmaceutically acceptable salt thereof, e.g., iptacopan hydrochloride, e.g., iptacopan hydrochloride monohydrate Form HB, for use in achieving a stable or improved eGFR, in a subject, e.g., a patient, in need thereof, wherein the treatment comprises administering to the subject, e.g., patient, iptacopan or a pharmaceutically acceptable salt thereof, e.g., iptacopan hydrochloride, e.g., iptacopan hydrochloride monohydrate Form HB.

In another aspect, the disclosure provides use of iptacopan or a pharmaceutically acceptable salt thereof, e.g., iptacopan hydrochloride, e.g., iptacopan hydrochloride monohydrate Form HB, in the manufacture of a medicament for the treatment of immune complex mediated membranoproliferative glomerulonephritis (IC-MPGN) in a subject, e.g., a patient, in need thereof, wherein the treatment comprises administering to the subject, e.g., patient, iptacopan or a pharmaceutically acceptable salt thereof, e.g., iptacopan hydrochloride, e.g., iptacopan hydrochloride monohydrate Form HB.

In another aspect, the disclosure provides use of iptacopan or a pharmaceutically acceptable salt thereof, e.g., iptacopan hydrochloride, e.g., iptacopan hydrochloride monohydrate Form HB, in the manufacture of a medicament for reducing proteinuria, e.g., reducing UPCR, in a subject, e.g., a patient, in need thereof, wherein the treatment comprises orally administering to the subject, e.g., patient, iptacopan or a pharmaceutically acceptable salt thereof, e.g., iptacopan hydrochloride, e.g., iptacopan hydrochloride monohydrate Form HB.

In another aspect, the disclosure provides use of iptacopan or a pharmaceutically acceptable salt thereof, e.g., iptacopan hydrochloride, e.g., iptacopan hydrochloride monohydrate Form HB, in the manufacture of a medicament for achieving a stable or improved eGFR, in a subject, e.g., a patient, in need thereof, wherein the treatment comprises orally administering to the subject, e.g., patient, iptacopan or a pharmaceutically acceptable salt thereof, e.g., iptacopan hydrochloride, e.g., iptacopan hydrochloride monohydrate Form HB.

In another aspect, the disclosure provides a pharmaceutical composition comprising iptacopan or a pharmaceutically acceptable salt thereof, e.g., iptacopan hydrochloride, e.g., iptacopan hydrochloride monohydrate Form HB, for use in treating immune complex mediated membranoproliferative glomerulonephritis (IC-MPGN), in a subject, e.g., patient, in need thereof, wherein the pharmaceutical composition is to be administered to thereby treat the subject, e.g., patient.

In another aspect, the disclosure provides a pharmaceutical composition comprising iptacopan or a pharmaceutically acceptable salt thereof, e.g., iptacopan hydrochloride, e.g., iptacopan hydrochloride monohydrate Form HB, for use in reducing proteinuria, e.g. reducing UPCR, in a subject, e.g., patient, in need thereof, wherein the pharmaceutical composition is to be administered, to thereby treat the subject, e.g., patient.

In another aspect, the disclosure provides a pharmaceutical composition comprising iptacopan or a pharmaceutically acceptable salt thereof, e.g., iptacopan hydrochloride, e.g., iptacopan hydrochloride monohydrate Form HB, for use in achieving a stable or improved eGFR, in a subject, e.g., patient, in need thereof, wherein the pharmaceutical composition is to be administered, to thereby treat the subject, e.g., patient.

The above aspects of the invention are each represented by the following embodiments which may be combined as applicable.

In an embodiment, iptacopan or a pharmaceutically acceptable salt thereof, e.g., iptacopan hydrochloride, e.g., iptacopan hydrochloride monohydrate Form HB, is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, administered twice daily (b.i.d.), e.g., about every 12 hours (wherein the dosing amount refers to the anhydrous free base of iptacopan hydrochloride). In another embodiment, iptacopan or a pharmaceutically acceptable salt thereof, e.g., iptacopan hydrochloride, e.g., iptacopan hydrochloride monohydrate Form HB, is administered at a dose of from 50 mg to 200 mg, e.g., from 50 mg to from 100 mg, from 100 mg to 200 mg, at a dose of 50 mg, 75 mg, 100 mg, 150 mg, or 200 mg, administered twice daily (b.i.d.), e.g., every 12 hours (wherein the dosing amount refers to the anhydrous free base of iptacopan hydrochloride).

In an embodiment, iptacopan or a pharmaceutically acceptable salt thereof, e.g., iptacopan hydrochloride, e.g., iptacopan hydrochloride monohydrate Form HB, is administered at a dose of about 100 mg twice daily (b.i.d.), in particular at a dose of 100 mg twice daily (b.i.d.) (wherein the dosing amount refers to the anhydrous free base of iptacopan hydrochloride).

In an embodiment, iptacopan or a pharmaceutically acceptable salt thereof, e.g., iptacopan hydrochloride, e.g., iptacopan hydrochloride monohydrate Form HB, is administered at a dose of about 200 mg twice daily (b.i.d.) (wherein the dosing amount refers to the anhydrous free base of iptacopan hydrochloride). In a preferred embodiment, iptacopan or a pharmaceutically acceptable salt thereof, e.g., iptacopan hydrochloride, e.g., iptacopan hydrochloride monohydrate Form HB, is administered at a dose of 200 mg twice daily (b.i.d.) (wherein the dosing amount refers to the anhydrous free base of iptacopan hydrochloride).

In an embodiment, iptacopan or a pharmaceutically acceptable salt thereof, e.g., iptacopan hydrochloride, e.g., iptacopan hydrochloride monohydrate Form HB, is administered orally.

In an embodiment, the method comprises administering, e.g., orally, to the subject, e.g., patient, iptacopan hydrochloride monohydrate Form HB.

In an embodiment, iptacopan hydrochloride monohydrate Form HB is a crystalline form characterized by the PXRD peaks identified at 2-Theta angles of (4.6±0.2)°, (9.2±0.2)°, and (19.1±0.2)°, when measured at a temperature in the range of from 20 to 30° C. with Cu-Kalpha1,2 radiation having a wavelength of 0.15419 nm.

In an embodiment, iptacopan hydrochloride monohydrate Form HB is a crystalline form characterized by the PXRD peaks identified at 2-Theta angles of (4.6±0.2)°, (6.8±0.2)°, (9.2±0.2)°, and (19.1±0.2)°, when measured at a temperature in the range of from 20 to 30° C. with Cu-Kalpha1,2 radiation having a wavelength of 0.15419 nm.

In an embodiment, iptacopan hydrochloride monohydrate Form HB is a crystalline form characterized by the PXRD peaks identified at 2-Theta angles of (4.6±0.2)°, (6.8±0.2)°, (9.2±0.2)°, (19.1±0.2)°, and (24.6±0.2)°, when measured at a temperature in the range of from 20 to 30° C. with Cu-Kalpha1,2 radiation having a wavelength of 0.15419 nm.

In an embodiment, iptacopan hydrochloride monohydrate Form HB is a crystalline form characterized by the PXRD peaks identified at 2-Theta angles of (4.6±0.2)°, (6.8±0.2)°, (9.2±0.2)°, (12.2±0.2)°, (19.1±0.2)°, and (24.6±0.2)°, when measured at a temperature in the range of from 20 to 30° C. with Cu-Kalpha1,2 radiation having a wavelength of 0.15419 nm.

In an embodiment, iptacopan hydrochloride monohydrate Form HB is a crystalline form characterized by the PXRD peaks identified at 2-Theta angles of (4.6±0.2)°, (6.8±0.2)°, (9.2±0.2)°, (12.2±0.2)°, (16.6±0.2)°, (19.1±0.2)°, (21.3±0.2)°, and (24.6±0.2)°, when measured at a temperature in the range of from 20 to 30° C. with Cu-Kalpha1,2 radiation having a wavelength of 0.15419 nm.

In an embodiment, iptacopan hydrochloride monohydrate Form HB is a crystalline form characterized by the PXRD peaks identified at 2-Theta angles of:
(4.6±0.1)°, (9.2±0.1)°, and (19.1±0.1)°; or
(4.6±0.1)°, (6.8±0.1)°, (9.2±0.1)°, and (19.1±0.1)°; or
(4.6±0.1)°, (6.8±0.1)°, (9.2±0.1)°, (19.1±0.1)°, and (24.6±0.1)°; or
(4.6±0.1)°, (6.8±0.1)°, (9.2±0.1)°, (12.2±0.1)°, (19.1±0.1)°, and (24.6±0.1)°; or
(4.6±0.1)°, (6.8±0.1)°, (9.2±0.1)°, (12.2±0.1)°, (19.1±0.1)°, (21.3±0.1)°, and (24.6±0.1)°; or
(4.6±0.1)°, (6.8±0.1)°, (9.2±0.1)°, (12.2±0.1)°, (12.6±0.1)°, (16.6±0.1)°, (19.1±0.1)°, (21.3±0.1)°, and (24.6±0.1)°; or
(4.6±0.1)°, (6.8±0.1)°, (9.2±0.1)°, (10.0±0.1)°, (12.2±0.1)°, (12.6±0.1)°, (16.6±0.1)°, (19.1±0.1)°, (21.3±0.1)°, and (24.6±0.1)°; or
(4.6±0.1)°, (6.8±0.1)°, (9.2±0.1)°, (10.0±0.1)°, (12.2±0.1)°, (12.6±0.1)°, (16.6±0.1)°, (19.1±0.1)°, (21.3±0.1)°, and (24.6±0.1)°; or
(4.6±0.1)°, (6.8±0.1)°, (9.2±0.1)°, (10.0±0.1)°, (12.2±0.1)°, (12.6±0.1)°, (15.3±0.1)°, (16.6±0.1)°, (19.1±0.1)°, (21.3±0.1)°, and (24.6±0.1)°; or
(4.6±0.1)°, (6.8±0.1)°, (9.2±0.1)°, (10.0±0.1)°, (12.2±0.2)°, (12.6±0.2)°, (15.3±0.2)°, (16.6±0.2)°, (17.2±0.2)°, (19.1±0.2)°, (21.3±0.2)°, and (24.6±0.2)°; or
(4.6±0.2)°, (6.8±0.2)°, (9.2±0.2)°, (10.0±0.2)°, (12.2±0.2)°, (12.6±0.2)°, (15.3±0.2)°, (16.6±0.2)°, (17.2±0.2)°, (19.1±0.2)°, (20.7±0.2)°, (21.3±0.2)°, and (24.6±0.2)°; or
(4.6±0.2)°, (6.8±0.2)°, (9.2±0.2)°, (10.0±0.2)°, (12.2±0.2)°, (12.6±0.2)°, (15.3±0.2)°, (16.6±0.2)°, (17.2±0.2)°, (19.1±0.2)°, (20.7±0.2)°, (21.3±0.2)°, (24.0±0.2)°, and (24.6±0.1)°; or
(4.6±0.1)°, (6.8±0.1)°, (9.2±0.1)°, (10.0±0.1)°, (12.2±0.1)°, (12.6±0.1)°, (15.3±0.1)°, (16.6±0.1)°, (17.2±0.1)°, (19.1±0.1)°, (20.7±0.1)°, (21.3±0.1)°, (22.2±0.1)°, (24.0±0.1)°, and (24.6±0.1)°; or
(4.6±0.1)°, (6.8±0.1)°, (9.2±0.1)°, (10.0±0.1)°, (12.2±0.2)°, (12.6±0.2)°, (15.3±0.1)°, (16.6±0.1)°, (17.2±0.1)°, (19.1±0.1)°, (20.7±0.1)°, (21.3±0.1)°, (22.2±0.1)°, (24.0±0.1)°, (24.6±0.1°) and (28.0±0.1)°, when measured at a temperature in the range of from 20 to 30° C. with Cu-Kalpha1,2 radiation having a wavelength of 0.15419 nm.

In an embodiment, iptacopan hydrochloride monohydrate Form HB is a crystalline form characterized by having a Fourier transform infrared spectrum comprising peaks at wavenumbers of comprising peaks at wavenumbers of (3452±4) cm⁻¹, (2875±4) cm⁻¹, and (1692±4) cm⁻¹, when measured at a temperature in the range of from 20° C. to 30° C. with a diamond ATR cell.

In an embodiment, iptacopan hydrochloride monohydrate Form HB is a crystalline form characterized by having a Fourier transform infrared spectrum comprising peaks at wavenumbers of (3452±4) cm⁻¹, (2875±4) cm⁻¹, (1692±4) cm⁻¹, and (1439±4) cm⁻¹, when measured at a temperature in the range of from 20° C. to 30° C. with a diamond ATR cell.

In an embodiment, iptacopan hydrochloride monohydrate Form HB is a crystalline form characterized by having a Fourier transform infrared spectrum comprising peaks at wavenumbers of:
(3452±2) cm⁻¹, (2875±2) cm⁻¹, and (1692±2) cm⁻¹, or
(3452±2) cm⁻¹, (2875±2) cm⁻¹, (1692±2) cm⁻¹, and (1439±2) cm⁻¹; or
(3452±2) cm⁻¹, (2875±2) cm⁻¹, (1692±2) cm⁻¹, (1439±2) cm⁻¹ and (1243±2) cm⁻¹; or
(3452±2) cm⁻¹, (2875±2) cm⁻¹, (1692±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1692±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1439±2) cm⁻¹, (1243±2) cm⁻¹ and (767±2) cm¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1384±2) cm⁻¹, (1243±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1384±2) cm⁻¹, (1243±2) cm⁻¹, (1184±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1384±2) cm⁻¹, (1243±2) cm⁻¹, (1184±2) cm⁻¹, (1069±2) cm⁻¹ and (767±2) cm⁻¹; or
(3452±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1384±2) cm⁻¹, (1243±2) cm⁻¹, (1184±2) cm⁻¹, (1069±2) cm⁻¹, (767±2) cm⁻¹ and (739±2) cm⁻¹; or
(3452±2) cm⁻¹, (3274±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1384±2) cm⁻¹, (1243±2) cm⁻¹, (1184±2) cm⁻¹, (1069±2) cm⁻¹, (767±2) cm⁻¹ and (739±2) cm⁻¹; or
(3452±2) cm⁻¹, (3274±2) cm⁻¹, (2933±2) cm⁻¹, (2875±2) cm⁻¹, (2732±2) cm⁻¹, (1709±2) cm⁻¹, (1692±2) cm⁻¹, (1658±2) cm⁻¹, (1615±2) cm⁻¹, (1601±2) cm⁻¹, (1515±2) cm⁻¹, (1497±2) cm⁻¹, (1461±2) cm⁻¹, (1439±2) cm⁻¹, (1425±2) cm⁻¹, (1384±2) cm⁻¹, (1243±2) cm⁻¹, (1184±2) cm⁻¹, (1069±2) cm⁻¹, (767±2) cm⁻¹ and (739±2) cm⁻¹, when measured at a temperature in the range of from 20 to 30° C. with a diamond ATR cell.

In an embodiment, iptacopan hydrochloride monohydrate Form HB is a crystalline form characterized by having a differential scanning calorimetry curve comprising an endothermic event in the range of from 35° C. to 170° C., when measured at a heating rate of 10 K/min.

In an embodiment, iptacopan hydrochloride monohydrate Form HB is a crystalline form characterized by having a DSC curve showing a broad endothermic event which ends at about 170° C., followed by exothermic decomposition at about 200° C., when measured at a heating rate of 10 K/min. In an embodiment, the broad endothermic event which ends at about 170° C., is an endothermic event in the range of 35° C. to 170° C. when measured at a heating rate of 10 K/min.

In an embodiment, iptacopan hydrochloride monohydrate Form HB is a crystalline form characterized by having a thermogravimetric analysis curve showing a mass loss at about 220° C., such as at a temperature of from 200 to 220° C., due to loss of water and residual solvents of not more than 4.5 w-%, e.g., of not more than 4.3 w-%, e.g., of not more than 4.0 w-%, e.g., of not more than 3.8 w-%, for example of not more than 3.4 w-%, based on the weight of the crystalline form, when heated from 30 to 300° C. at a rate of 20 K/min.

In an embodiment, iptacopan hydrochloride monohydrate Form HB is a crystalline form characterized by having a dynamic vapor sorption curve showing a mass change of not more than 4.5 w-%, e.g., of not more than 4.0 w-%, e.g., of not more than 3.0 w-%, e.g., of not more than 2.0 w-%, for example of not more than 1.8 w %, 1.6 w-%, 1.5 w-% or 1.4 w-%, based on the weight of the crystalline form, when measured with dynamic vapor sorption at a relative humidity in the range of from 0 to 95% and a temperature of (25±1.0° C.)

In an embodiment, the subject, e.g., patient, is an adult. In one embodiment, the subject, e.g., patient, is an adolescent.

In an embodiment, the subject, e.g., patient, is diagnosed as having IC-MPGN, e.g., primary IC-MPGN, e.g., in native kidneys, e.g., as confirmed by kidney biopsy, e.g., within 12 months in adults or within 3 years in adolescents prior to administering.

In an embodiment, the subject, e.g., patient, has a urine protein/creatinine ratio (UPCR)≥1.0 g/g, e.g., sampled from a first morning void or 24 hour urine collection, prior to administering iptacopan or a pharmaceutically acceptable salt thereof, e.g., iptacopan hydrochloride, e.g., iptacopan hydrochloride monohydrate Form HB.

In an embodiment, the subject, e.g., patient, has an estimated glomerular filtration rate (eGFR)≥30 ml/min/1.73 m², e.g., calculated using the CKD-EPI formula or modified Schwartz formula according to specific ethnic groups and local practice guidelines, prior to administering iptacopan or a pharmaceutically acceptable salt thereof, e.g., iptacopan hydrochloride, e.g., iptacopan hydrochloride monohydrate Form HB.

In an embodiment, the subject, e.g., patient, has a measured glomerular filtration rate (GFR)≥30 ml/min/1.73 m², prior to administering iptacopan or a pharmaceutically acceptable salt thereof, e.g., iptacopan hydrochloride, e.g., iptacopan hydrochloride monohydrate Form HB.

In an embodiment, the subject, e.g., patient, has been vaccinated prior to administering iptacopan or a pharmaceutically acceptable salt thereof, e.g., iptacopan hydrochloride, e.g., iptacopan hydrochloride monohydrate Form HB.

In an embodiment, the subject, e.g., patient, has been vaccinated against encapsulated bacteria prior to administering iptacopan or a pharmaceutically acceptable salt thereof, e.g., iptacopan hydrochloride, e.g., iptacopan hydrochloride monohydrate Form HB.

In an embodiment, the subject, e.g., patient, has been or is administered antibiotics, e.g., against encapsulated bacteria.

In an embodiment, the encapsulated bacteria are Neisseria meningitidis, Streptococcus pneumoniae, Haemophilus influenzae, or Haemophilus influenzae.

In an embodiment, the subject, e.g., patient, has been vaccinated against Neisseria meningitidis (types A, C, Y and W-135) prior to administering iptacopan or a pharmaceutically acceptable salt thereof e.g., iptacopan hydrochloride, e.g., iptacopan hydrochloride monohydrate Form HB.

In an embodiment, the subject, e.g., patient, has been vaccinated against Streptococcus pneumoniae (Pneumovax-23) prior to administering iptacopan or a pharmaceutically acceptable salt thereof e.g., iptacopan hydrochloride, e.g., iptacopan hydrochloride monohydrate Form HB.

In an embodiment, the subject, e.g., patient, has been vaccinated against Haemophilus influenzae prior to administering iptacopan or a pharmaceutically acceptable salt thereof, e.g., iptacopan hydrochloride, e.g., iptacopan hydrochloride monohydrate Form HB.

In an embodiment, the subject, e.g., patient, has been administered a supportive care, e.g., at a maximal daily dose or maximally tolerated dose, e.g., at a stable dose, e.g., for at least 90 days prior to administering iptacopan or a pharmaceutically acceptable salt thereof.

In an embodiment, the supportive care is at a maximal daily dose or maximally tolerated dose.

In an embodiment, the supportive care is at a stable dose.

In an embodiment, the supportive care has been administered for at least 90 days prior to treatment.

In an embodiment, the subject, e.g., patient, is further administered a supportive care, e.g., at a stable dose.

In an embodiment, the subject, e.g., patient, has been administered and is being further administered an antiproteinuric medication.

In an embodiment, the subject, e.g., patient, has been administered and is being further administered an immunosuppressant.

In an embodiment, the supportive care comprises a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor (e.g., voclosporin).

In an embodiment, the subject, e.g., patient, has been administered and is being further administered a/an ACEi, ARB, mycophenolic acid, corticosteroid, SGLT2 inhibitor, mineralocorticoid receptor antagonist, cyclophosphamide (CYC), or calcineurin inhibitor (e.g., voclosporin).

In an embodiment, the subject, e.g., patient, has been administered and is being further administered a/an ACEi or ARB, e.g., at a stable maximally-tolerated dose.

In an embodiment, the subject, e.g., patient, has been administered and is being further administered mycophenolate mofetil (MMF) or mycophenolate sodium (MPS).

In an embodiment, the subject, e.g., patient, has been administered and is being further administered an immunosuppressant or antiproteinuric medications, e.g., MMF, MPS, corticosteroids and SGLT2i, at a stable dose.

In an embodiment, the subject, e.g., patient, has been administered and is being further administered a corticosteroid, e.g., prednisone, e.g., at no greater than 7.5 mg per day.

In an embodiment, the disclosure provides iptacopan or a pharmaceutically acceptable salt thereof, e.g., iptacopan hydrochloride, e.g., iptacopan hydrochloride monohydrate Form HB, for use in reducing proteinuria, e.g., reducing UPCR, in a subject, e.g., a patient, wherein the UPCR in a subject, e.g., a patient, is reduced, e.g., by no less than 25%, no less than 30%, no less than 40%, no less than 45%, no less than 50%, no less than 55%, no less than 60% or no less than 65%, compared to prior to administering iptacopan or a pharmaceutically acceptable salt thereof, e.g., iptacopan hydrochloride, e.g., iptacopan hydrochloride monohydrate Form HB. In an embodiment, the UPCR in a subject, e.g., a patient, is reduced, e.g., by no less than 25%, no less than 30%, no less than 40%, no less than 45%, no less than 50%, no less than 55%, no less than 60% or no less than 65%, after 6 months of treatment compared to prior to administering iptacopan or a pharmaceutically acceptable salt thereof, e.g., iptacopan hydrochloride, e.g., iptacopan hydrochloride monohydrate Form HB. In a specific embodiment, iptacopan or a pharmaceutically acceptable salt thereof, e.g., iptacopan hydrochloride, e.g., iptacopan hydrochloride monohydrate Form HB, is administered at a dose of 200 mg twice daily (b.i.d.) (wherein the dosing amount refers to the anhydrous free base of iptacopan hydrochloride).

In an embodiment, the disclosure provides a method of achieving a stable or improved estimated glomerular filtration rate (eGFR) in a subject, e.g., a patient, in need thereof, the method comprising administering, e.g., orally, to the subject, e.g., patient, iptacopan or a pharmaceutically acceptable salt thereof, e.g., iptacopan hydrochloride, e.g., iptacopan hydrochloride monohydrate Form HB, wherein the eGFR or measured GFR in a subject, e.g., a patient, is stable, e.g., no greater than 20% reduction, no greater than 15% reduction, no greater than 10% reduction, or no greater than 5% reduction, compared to prior to administering iptacopan or a pharmaceutically acceptable salt thereof, e.g., iptacopan hydrochloride, e.g., iptacopan hydrochloride monohydrate Form HB.

In an embodiment, the eGFR or measured GFR in a subject, e.g., a patient, is stable, e.g., no greater than 20% reduction, no greater than 15% reduction, no greater than 10% reduction, or no greater than 5% reduction, after 6 months of treatment compared to prior to administering iptacopan or a pharmaceutically acceptable salt thereof, e.g., iptacopan hydrochloride e.g., iptacopan hydrochloride monohydrate Form HB.

In an embodiment, the disclosure provides a method of achieving a stable or improved estimated glomerular filtration rate (eGFR) in a subject, e.g., a patient, in need thereof, the method comprising administering, e.g., orally, to the subject, e.g., patient, iptacopan or a pharmaceutically acceptable salt thereof, e.g., iptacopan hydrochloride, e.g., iptacopan hydrochloride monohydrate Form HB, wherein the eGFR or measured GFR in a subject, e.g., a patient, is improved, compared to prior to administering iptacopan or a pharmaceutically acceptable salt thereof, e.g., iptacopan hydrochloride, e.g., iptacopan hydrochloride monohydrate Form HB. In an embodiment, the eGFR or measured GFR in a subject, e.g., a patient, is improved, after 6 months of treatment compared to prior to administering iptacopan or a pharmaceutically acceptable salt thereof, e.g., iptacopan hydrochloride.

In an embodiment, the disclosure provides iptacopan or a pharmaceutically acceptable salt thereof, e.g., iptacopan hydrochloride, for use in reducing proteinuria, e.g., reducing UPCR, in a subject, e.g., a patient, in need thereof, wherein the treatment comprises administering to the subject, e.g., patient, iptacopan or a pharmaceutically acceptable salt thereof, e.g., iptacopan hydrochloride, wherein the UPCR in a subject, e.g., a patient, is reduced, e.g., by no less than 25%, no less than 30%, no less than 40%, no less than 45%, no less than 50%, no less than 55%, no less than 60% or no less than 65%; and the eGFR or measured GFR in a subject, e.g., a patient, is stable, e.g., no greater than 20% reduction, no greater than 15% reduction, no greater than 10% reduction, or no greater than 5% reduction, compared to prior to administering iptacopan or a pharmaceutically acceptable salt thereof, e.g., iptacopan hydrochloride, e.g., iptacopan hydrochloride monohydrate Form HB. In an embodiment, the UPCR in a subject, e.g., a patient, is reduced, e.g., by no less than 25%, no less than 30%, no less than 40%, no less than 45%, no less than 50%, no less than 55%, no less than 60% or no less than 65%; and the eGFR or measured GFR in a subject, e.g., a patient, is stable, e.g., no greater than 20% reduction, no greater than 15% reduction, no greater than 10% reduction, or no greater than 5% reduction, after 6 months of treatment compared to prior to administering iptacopan or a pharmaceutically acceptable salt thereof, e.g., iptacopan hydrochloride.

In an embodiment, a FACIT-Fatigue score of the subject, e.g., patient, is reduced compared to prior to administering iptacopan or a pharmaceutically acceptable salt thereof, e.g., iptacopan hydrochloride.

In an embodiment, the disclosure provides a method of treating immune complex mediated membranoproliferative glomerulonephritis (IC-MPGN) in a subject, e.g., a patient, in need thereof, the method comprising administering, e.g., orally, to the subject, e.g., patient, iptacopan or a pharmaceutically acceptable salt thereof, e.g., iptacopan hydrochloride, to thereby treat the subject, e.g., patient, wherein a FACIT-Fatigue score of the subject, e.g., patient, is reduced by no less than 2, no less than 3, or no less than 4 points, compared to prior to administering iptacopan or a pharmaceutically acceptable salt thereof, e.g., iptacopan hydrochloride, e.g., iptacopan hydrochloride monohydrate Form HB. In an embodiment, a FACIT-Fatigue score of the subject, e.g., patient, is reduced by no less than 2, no less than 3, or no less than 4 points, after 6 months, or after 12 months of treatment compared to prior to administering iptacopan or a pharmaceutically acceptable salt thereof, e.g., iptacopan hydrochloride, e.g., iptacopan hydrochloride monohydrate Form HB.

### Key Efficacy Assessment

Also provided herein is a method of assessing the efficacy of treatment in a patient population with immune complex mediated membranoproliferative glomerulonephritis (IC-MPGN) treated with iptacopan or a pharmaceutically acceptable salt thereof, e.g., iptacopan hydrochloride, e.g., iptacopan hydrochloride monohydrate Form HB, the method comprising determining the percentage of the patient population achieving proteinuria reduction as compared to a patient population untreated with iptacopan or a pharmaceutically acceptable salt thereof, e.g., iptacopan hydrochloride, e.g., iptacopan hydrochloride monohydrate Form HB, to thereby assess efficacy of treatment.

Also provided herein is a method of assessing the efficacy of treatment in a patient population with immune complex mediated membranoproliferative glomerulonephritis (IC-MPGN) treated with iptacopan or a pharmaceutically acceptable salt thereof, e.g., iptacopan hydrochloride, e.g., iptacopan hydrochloride monohydrate Form HB, the method comprising determining the percentage of the patient population achieving a stable or improved eGFR as compared to a patient population untreated with iptacopan or a pharmaceutically acceptable salt thereof, e.g., iptacopan hydrochloride, e.g., iptacopan hydrochloride monohydrate Form HB, to thereby assess efficacy of treatment.
Efficacy assessment:
   A stable or improved eGFR compared to the baseline visit (≤15% reduction in eGFR), and/or
   ≥50% reduction in urine protein/creatinine ratio (UPCR)
Patient population: participants with biopsy-confirmed idiopathic IC-MPGN and meet other inclusion and exclusion criteria
A Proportion of patients achieving the following criteria compared to the baseline visit:
   A stable or improved eGFR compared to the baseline visit (150 reduction in eGFR), and
   ≥50%; reduction in urine protein/creatinine ratio (UPCR)

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Glossary of Terms

| | |
|---|---|
| ACEI/ARB | Angiotensin converting enzyme inhibitors/Angiotensin |
| | receptor blockers is class of medication recommended by |
| | KDIGO guideline for treatment of IC-MPGN ATC-codes: |
| | C09; combinations with other medicines under C10BX |
| Additional | Medicinal products that may be used during the clinical trial |
| treatment | as described in the protocol, but not as an investigational |
| | medicinal product (e.g. any background therapy) |
| Assessment | A procedure used to generate data required by the study |
| Biologic samples | A biological specimen including, for example, blood |
| | (plasma, serum), saliva, tissue, urine, stool, etc. taken from a |
| | study participant |
| Cohort | A specific group of participants fulfilling certain criteria and |
| | generally treated at the same time |
| Control drug | A study drug (active or placebo) used as a comparator to |
| | reduce assessment bias, preserve blinding of investigational |
| | drug, assess internal study validity, and/or evaluate |
| | comparative effects of the investigational drug |
| Dosage | Dose of the study treatment given to the participant in a time |
| | unit (e.g. 100 mg once a day, 75 mg twice a day) |
| Electronic Data | Electronic data capture (EDC) is the electronic acquisition of |
| Capture (EDC) | clinical study data using data collection systems, such as |
| | Web-based applications, interactive voice response systems |
| | and clinical laboratory interfaces. EDC includes the use of |
| | Electronic Case Report Forms (eCRFs) which are used to |
| | capture data transcribed from paper source forms used at the |
| | point of care |
| End of the | The end of the clinical trial is defined as the last visit of the |
| clinical trial | last participant or at a later point in time as defined by the |
| | protocol |
| Enrollment | Point/time of participant entry into the study at which |
| | informed consent must be obtained |
| Estimand | A precise description of the treatment effect reflecting the |
| | clinical question posed by the trial objective. It summarizes |
| | at a population-level what the outcomes would be in the same |
| | patients under different treatment conditions being |
| | compared. Attributes of an estimand include the population, |
| | variable (or endpoint) and treatment of interest, as well as the |
| | specification of how the remaining intercurrent events are |
| | addressed and a population-level summary for the variable. |
| Glucocorticoids/ | As per KDIGO 2021, glucocorticoids can be added to the |
| Corticosteroids | supportive therapy for patients with idiopathic immune |
| | complex-mediated glomerulonephritis (ICGN), abnormal |
| | kidney function (but without crescentic involvements), |
| | active urine sediment, with or without nephrotic-range |
| | proteinuria. ATC code- H02 |
| Healthy volunteer | A person with no known significant health problems who |
| | volunteers to be a study participant |
| Intercurrent events | Events occurring after treatment initiation that affect either |
| | the interpretation or the existence of the measurements |
| | associated with the clinical question of interest. |
| Investigational drug/treatment | The drug whose properties are being tested in the study |
| Medication number | A unique identifier on the label of medication kits |
| Mis-randomized participants | Mis-randomized participants are those who were not |
| | qualified for randomization and who did not take study |
| | treatment, but have been inadvertently randomized into the |
| | study |
| Other treatment | Treatment that may be needed/allowed during the conduct of |
| | the study (i.e. concomitant or rescue therapy) |
| Part | A sub-division of a study used to evaluate specific objectives |
| | or contain different populations. For example, one study |
| | could contain a single dose part and a multiple dose part, or |
| | a part in participants with established disease and in those |
| | with newly-diagnosed disease |
| Participant | A trial participant (can be a healthy volunteer or a patient) |
| Participant number | A unique number assigned to each participant upon signing |
| | the informed consent. This number is the definitive, unique |
| | identifier for the participant and should be used to identify |
| | the participant throughout the study for all data collected, |
| | sample labels, etc. |
| Period | The subdivisions of the trial design (e.g. Screening, |
| | Treatment, Follow-up) which are described in the Protocol. |
| | Periods define the study phases and will be used in clinical |
| | trial database setup and eventually in analysis |
| Personal data | Participant information collected by the Investigator that is |
| | coded and transferred to Novartis for the purpose of the |
| | clinical trial. This data includes participant identifier |
| | information, study information and biological samples. |
| Premature participant | Point/time when the participant exits from the study prior to |
| withdrawal | the planned completion of all study drug administration |
| | and/or assessments; at this time all study drug administration |
| | is discontinued and no further assessments are planned |
| Randomization | The process of assigning trial participants to investigational |
| | drug or control/comparator drug using an element of chance |
| | to determine the assignments in order to reduce bias. |
| Randomization number | A unique identifier assigned to each randomized participant |
| Screen Failure | A participant who did not meet one or more criteria that were |
| | required for participation in the study |
| SGLT2 inhibitors | Sodium-glucose co-transporter 2 (SGLT2) inhibitors is a |
| | class of medications used in diabetes with nephroprotective |
| | effects recently demonstrated in large outcome trials in |
| | diabetic and non-diabetic chronic kidney disease (CKD). |
| | Some of these medications maybe used as supportive therapy |
| | in IC-MPGN. |
| | ATC code: A10BK |
| Source Data/Document | Source data refers to the initial record, document, or primary |
| | location from where data comes. The data source can be a |
| | database, a dataset, a spreadsheet or even hard-coded data, |
| | such as paper or eSource |
| Start of the clinical | The start of the clinical trial is defined as the signature of the |
| trial | informed consent by the first participant |
| Study treatment | Any drug or combination of drugs or intervention |
| | administered to the study participants as part of the required |
| | study procedures; includes investigational drug(s), control(s) |
| | or background therapy |
| Study treatment | When the participant permanently stops taking any of the |
| discontinuation | study drug(s) prior to the defined study treatment completion |
| | date (if any) for any reason; may or may not also be the |
| | point/time of study discontinuation |
| Treatment arm/group | A treatment arm/group defines the dose and regimen or the |
| | combination, and may consist of 1 or more cohorts. |
| Treatment of interest | The treatment of interest and, as appropriate, the alternative |
| | treatment to which comparison will be made. These might be |
| | individual interventions, combinations of interventions |
| | administered concurrently, e.g. as add-on to standard of care, |
| | or might consist of an overall regimen involving a complex |
| | sequence of interventions. This is the treatment of interest |
| | used in describing the related clinical question of interest, |
| | which might or might not be the same as the study treatment. |
| Variable (or endpoint) | The variable (or endpoint) to be obtained for each participant |
| | that is required to address the clinical question. The |
| | specification of the variable might include whether the |
| | participant experiences an intercurrent event. |
| Withdrawal of study | Withdrawal of consent from the study occurs only when a |
| consent (WoC) | participant does not want to participate in the study any |
| | longer and does not allow any further collection of personal |
| | data |

### Example 1. A Multicenter, Randomized, Double-Blind, Parallel Group, Placebo-Controlled Study to Evaluate the Efficacy and Safety of Iptacopan (LNP023) in Idiopathic Immune Complex Mediated Membranoproliferative Glomerulonephritis (IC-MPGN)

### Purpose

The purpose of this Phase III study (CLNP023B12302) is to evaluate the efficacy and safety of iptacopan compared to placebo (both administered in combination with standard of care) in participants (adults and adolescents aged 12-17 years) with idiopathic IC-MPGN. The study aims to demonstrate a reduction in proteinuria and improvement in estimated glomerular filtration rate (eGFR) in participants treated with iptacopan compared to placebo. Change in patient-reported fatigue will also be evaluated. Alternative complement pathway (AP) dysregulation is believed to underlie the clinical manifestations and progression of IC-MPGN.

### Study Design

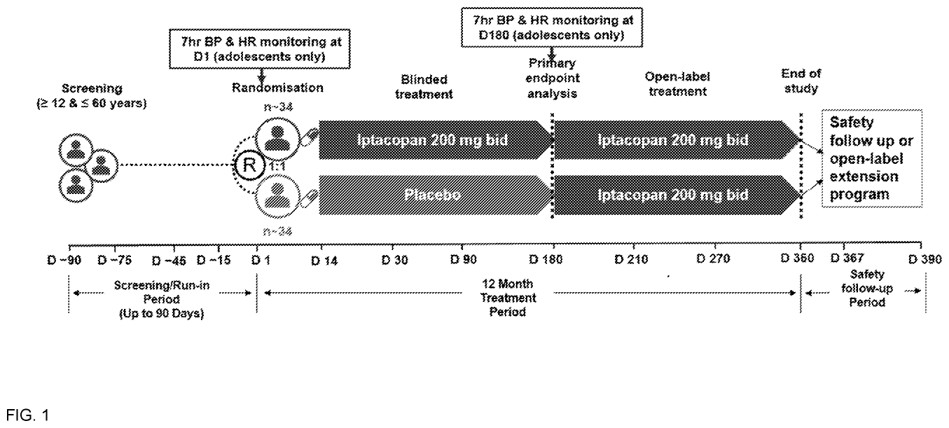

The study is a multicenter, randomized, double-blind, parallel group, placebo-controlled study to evaluate the efficacy and safety of iptacopan in participants with idiopathic IC-MPGN. Approximately 68 participants (adults and minimum of 10 adolescents) will be randomized in the trial. One half of the participants (n=34) will receive iptacopan at 200 mg bis in die/twice a day (b.i.d.) as blinded treatment for 6 months followed by 6 months of open-label iptacopan at 200 mg b.i.d. The other half of participants (n=34) will receive placebo as blinded treatment for 6 months followed by 6 months of open-label iptacopan at 200 mg b.i.d. Upon completion of study treatment at 12 months in CLNP023B12302, participants will have the option to discontinue iptacopan treatment and enter a 30-day Safety Follow-up period or transition to an open label extension study (CLNP023B12001B) and continue iptacopan treatment. The study design is demonstrated in FIG. 1.

The study comprises the following three periods:
Screening/run-in period of up to 90 days (note: it may be possible to complete the Screening/run-in period in less than 90 days, if for example the participant is already on a stable dose of antiproteinuric therapy (e.g. but not exclusively, angiotensin converting enzyme inhibitors, angiotensin II receptor blockers, SGLT2 inhibitors, mineralocorticoid receptor antagonists, mycophenolic acids, oral corticosteroids) and has already received all required vaccinations),
360-day Treatment period (including a 6-month blinded treatment part and a 6-month open-label part), and
30-day Safety follow-up period (or optionally, transition to an extension study).

Throughout the protocol, “baseline” refers to the Day 1 visit (pre-dose) of CLNP023B12302. Approximately 68 adult and adolescent participants (34 treated with iptacopan and 34 treated with placebo) aged 12-60 years, will be randomized. Minimum of 10 adolescents will be randomized separately in the study. Randomization will be stratified by corticosteroid and/or mycophenolic acid treatment at randomization (yes vs. no) in adult participants, since the use of these agents could affect the responsiveness to iptacopan.

In order to assess the efficacy of iptacopan in adult participants with high level proteinuria, approximately 50% of randomized participants are expected to have proteinuria≥3.0 g/day at Day 1 (24-hour urine collection). Baseline proteinuria will be monitored throughout the study and if necessary, randomization of adult participants may be restricted only to those with UPCR≥3.0 g/g at Day −75 and Day −15.

### Inclusion Criteria

Participants eligible for inclusion in this study must meet all of the following criteria:
1. Signed informed consent must be obtained prior to participation in the study.
2. Male and female participants age≥12 and ≤60 years at screening.
3. Able to communicate well with the investigator, understand and comply with the requirements of the study.
4. Diagnosis of IC-MPGN as confirmed by kidney biopsy within 12 months prior to enrollment in adults and within 3 years of enrollment in adolescents (a biopsy report, review and confirmation by the Investigator is required). If this confirmation is not available for an adult, it should be obtained by kidney biopsy at screening (adults only).
5. Prior to randomization, all participants must have been on a maximally recommended or tolerated dose of an angiotensin converting enzyme inhibitor (ACEI) or angiotensin receptor blocker (ARB) for at least 90 days (or as according to local guidelines). The doses of other antiproteinuric medications including mycophenolic acids (MMF or mycophenolate sodium), corticosteroids, SGLT2 inhibitors and mineralocorticoid receptor antagonists should be stable for at least 90 days prior to randomization. The doses of ACEI/ARB, mineralocorticoid receptor antagonists, mycophenolic acids, corticosteroids and SGLT2 inhibitors should remain stable throughout the study treatment period unless an adverse event or other safety issue requiring a change in dose is experienced by the participant.
6. UPCR≥1.0 g/g (≥113 mg/mmol) sampled from the first morning void urine sample at both Day −75 and Day −15, for adults and adolescents.
7. Estimated GFR (using the CKD-EPI formula for ≥18 years and modified Schwartz formula for 12 to 17 years) or measured GFR≥30 ml/min/1.73 m² at Screening and Day −15.
8. Mandatory vaccination against Neisseria meningitidis and Streptococcus pneumoniae infection is required prior to the start of study treatment. If the patient has not been previously vaccinated or if a booster is required, the vaccine should be given according to local regulations at least 2 weeks prior to the first administration of study treatment. If study treatment has to start earlier than 2 weeks post vaccination, prophylactic antibiotic treatment should be initiated in accordance with local standard of care.
9. If not previously vaccinated or if a booster is required, vaccination against Haemophilus influenzae infections should be given, if available and according to local regulations, at least 2 weeks prior to the first study treatment administration. If study treatment has to start earlier than 2 weeks post vaccination, prophylactic antibiotic treatment should be initiated in accordance with local standard of care.

### Key Exclusion Criteria

Participants meeting any of the following criteria are not eligible for inclusion in this study.
1. Participants who have received any cell or organ transplantation, including kidney transplantation.
2. Patients diagnosed with secondary IC-MPGN including but not limited to any of the following conditions:
   Deposition of antigen-antibody immune complexes as a result of any infection, including
      Viral—hepatitis C including HCV-associated mixed cryoglobulinemia, hepatitis B;
      Bacterial—endocarditis, infected ventriculo-atrial shunt, visceral abscesses, leprosy, meningococcal meningitis; chronic bacterial infections
      Protozoa/other infections—malaria, schistosomiasis, mycoplasma, leishmaniasis, filariasis, histroplasmosis)
   Deposition of immune complexes as a result of an autoimmune disease:
      SLE
      Sjogren's syndrome
      Rheumatoid arthritis
      Mixed connective tissue disease, etc
   Disposition of monoclonal Ig because of a monoclonal gammopathy due to plasma cell or B cell disorders. Monoclonal gammopathy of undetermined significance (MGUS) confirmed by the measurement of serum free light chains or other investigation as per local standard of care.
      Fibrillary glomerulonephritis
3. Rapidly progressive crescentic glomerulonephritis (defined as a 50% decline in the eGFR within 3 months) with renal biopsy findings of glomerular crescent formation seen in at least 50% of glomeruli.
4. Participants with acute post-infectious glomerulonephritis.
5. Renal biopsy showing interstitial fibrosis/tubular atrophy (IF/TA) of more than 50%.
6. Adult participants (≥18 and ≤60 years) with systolic blood pressure (SBP)<80 mmHg or >160 mmHg, or diastolic blood pressure (DBP)<50 mmHg or >100 mmHg, or pulse rate<45 bpm or >100 bpm.
7. Adolescent participants (ages 12-17 years) with systolic blood pressure (SBP)<80 mmHg or >150 mmHg, or diastolic blood pressure (DBP)<50 mmHg or >95 mmHg, or pulse rate<50 bpm or >110 bpm.
8. Body mass index (BMI)>38 kg/m² at screening and randomization. Body weight<35 kg at screening and randomization.
9. Participants with an active systemic bacterial, viral or fungal infection within 14 days prior to study treatment administration.
10. The presence of fever≥38° C. (100.4° F.) within 7 days prior to study treatment administration.
11. A history of recurrent invasive infections caused by encapsulated organisms, e.g., N. meningitidis and S. pneumoniae.
12. Human immunodeficiency virus (HIV) infection (known history of HIV or test positive for HIV antibody at screening).
13. Liver disease, such as active hepatitis B virus (HBV) or hepatitis C virus (HCV) infection defined as hepatitis B virus surface antigen (HBsAg) positive or hepatitis C virus ribonucleic acid (HCV RNA) positive, or liver injury as indicated by abnormal liver function tests at screening as defined below:
   Any single parameter of alanine aminotransferase (ALT), aspartate aminotransferase (AST), gamma-glutamyl transferase (GGT) or alkaline phosphatase must not exceed 3×upper limit of normal (ULN)
   Serum bilirubin must not exceed 2× upper limit of normal (ULN) (with the exception of those participants with a known confirmed diagnosis of Gilbert syndrome).
14. Evidence of urinary obstruction or difficulty in voiding at screening and randomization.
15. Severe concurrent comorbidities including but not limited to advanced cardiac disease (e.g., New York Heart Association [NYHA] class IV), severe pulmonary disease (e.g., severe pulmonary hypertension (World Health Organization [WHO] class IV)) or any other condition that in the opinion of the investigator precludes the participant's involvement in the study.
16. History or current diagnosis of ECG abnormalities indicating significant risk of safety for participants participating in the study such as clinically significant cardiac arrhythmias, e.g., sustained ventricular tachycardia and clinically significant second or third degree atrioventricular block (AV block) without a pacemaker.
17. History of malignancy of any organ system (other than localized basal cell carcinoma of the skin or in situ cervical cancer), treated or untreated, within the past 5 years, regardless of whether there is evidence of local recurrence or metastases.
18. History of hypersensitivity to any of the study treatments or excipients or to drugs of similar chemical classes or history of medium to high severity allergies, as per Investigator's judgement.
19. History of drug or alcohol abuse within the 12 months prior to study treatment administration.
20. Any medical condition deemed likely to interfere with the patient's participation in the study.
21. The use of inhibitors of complement factors (e.g., Factor B, Factor D, C3 inhibitors, anti C5 antibodies, C5a receptor antagonists) within 6 months prior to the Screening visit.
22. Participation in any other investigational drug trial or use of other investigational drugs at the time of enrollment, or within 5 elimination half-lives of enrollment, or within 30 days, whichever is longer; or longer if required by local regulations.
23. Female participants who are pregnant or breastfeeding or intending to conceive during the course of the study.
24. Women of child-bearing potential, defined as all women physiologically capable of becoming pregnant, unless they are using effective methods of contraception during dosing of investigational drug and for 1 week after stopping of investigational drug.

### Treatment Groups

The study comprises the following three periods (FIG. 1): Screening/run-in period of up to 90 days; 360-day Treatment period (including a 6-month blinded treatment part and a 6-month open-label part), and 30-day Safety follow-up period (or optionally, transition to an extension study). The duration of treatment with study drug is 12 months.

During the first 6 months of the double-blind treatment period, half of the participants will receive iptacopan 200 mg bid (as 100 mg for adolescents and 200 mg for adults) capsules. The other half of the participants will receive the placebo capsules matching those for 100 mg and 200 mg capsules of iptacopan in placebo control group. In the open-label period of 6 months, all participants in both groups receive iptacopan 200 mg bid. Iptacopan maybe temporarily interrupted or discontinued due to an AE, progression of underlying disease and/or at the discretion of the investigator or the participant.

### OBJECTIVES AND ENDPOINTS

TABLE 1
Objectives and related endpoints for double-blind treatment period

| Objective(s) | Endpoint(s) |
|---|---|
| Primary objective(s) | Endpoint(s) for primary objective(s) |
| To demonstrate the superiority of iptacopan | Log-transformed ratio to baseline in UPCR |
| compared to placebo in reducing proteinuria | (sampled from a 24-hour urine collection) at 6 |
| at 6 months of treatment. | months. |
| Secondary objective(s) | Endpoint(s) for secondary objective(s) |
| To demonstrate the superiority of iptacopan | Change from baseline in eGFR at 6 |
| vs. placebo in improving eGFR. | months. |
| To demonstrate the superiority of iptacopan | A participant meets the requirements of the |
| vs. placebo in the proportion of participants | composite renal endpoint if he/she satisfies |
| who achieved a composite renal endpoint. | the following criteria at the 6-month time |
| | point: (1) a stable or improved eGFR |
| | compared to the baseline visit (≤15% |
| | reduction in eGFR), and (2) a ≥50% |
| | reduction in UPCR compared to the baseline |
| | visit. Initiation of treatment with any |
| | complement pathway modifying agent or |
| | initiation/intensification of corticosteroid or |
| | immunosuppressant or renal replacement |
| | therapy automatically designates the |
| | participant as not meeting the endpoint. |
| To demonstrate the superiority of iptacopan | Change from baseline to 6 months in the |
| compared to placebo in improvement of | Functional Assessment of Chronic Illness |
| participant-reported fatigue. | Therapy-Fatigue (FACIT-Fatigue) score. |
| To evaluate the safety and tolerability of | Occurrence of clinically significant vital |
| iptacopan compared to placebo during the 6- | signs (msDBP, msSBP, heart rate), ECGs, |
| month double-blind period. | and safety laboratory measurements, as well |
| | as adverse events (AEs), AEs of special |
| | interest, and study drug discontinuation due to |
| | an AE (or any safety issue) during the double- |
| | blind period of the study. |
| To evaluate the effect of iptacopan compared | Occurrence of clinically significant |
| to placebo on blood pressure, heart rate, | changes in heart rate, msDBP/msSBP (on-site |
| cardiac structure and function and biomarkers | as well as home monitoring), |
| of cardiac injury during the 6-month double- | echocardiography parameters and NT- |
| blind period in adolescent participants. | proBNPin adolescent participants. |

TABLE 2
Objectives and related endpoints for open-label treatment period

| Objective(s) | Endpoint(s) |
|---|---|
| Primary objective(s) | Endpoint(s) for primary objective(s) |
| To evaluate the effect of iptacopan on | Log-transformed ratio to baseline in UPCR |
| proteinuria at 12 months. | at the 12-month visit (both study treatment |
| | arms). |
| | Log-transformed ratio to 6-month visit in |
| | UPCR at the 12-month visit in the placebo |
| | arm. |
| Secondary objective(s) | Endpoint(s) for secondary objective(s) |
| To evaluate the effect at 12 months of | 1. Change from baseline in eGFR at 12 |
| iptacopan in improving eGFR, on a composite | months (both study treatment arms). |
| renal endpoint, and in improvement of | 2. Change in eGFR from the 6-month visit to |
| participant-reported fatigue. | the 12-month visit of the placebo arm. |
| | 3. A participant is defined as meeting the |
| | requirements of the composite renal endpoint |
| | if they satisfy the eGFR and UPCR criteria at |
| | the 12-month time point (both treatment |
| | arms). The rate of this endpoint will also be |
| | evaluated in the placebo arm at the 12-month |
| | visit compared to the 6-month visit. |
| | 4. Change from baseline in the FACIT- |
| | Fatigue score at 12 months (both study |
| | treatment arms). |
| | 5. Change in the FACIT-Fatigue score from |
| | the 6-month visit to the 12-month visit of the |
| | placebo arm. |
| To evaluate the safety and tolerability of | 1. Occurrence of clinically significant vital |
| iptacopan during the 6-month open-label | signs (msDBP, msSBP, heart rate), ECGs, |
| treatment period as well as the entire 12- | and safety laboratory measurements, as well |
| month treatment period. | as adverse events (AEs), AEs of special |
| | interest, and study drug discontinuation due to |
| | an AE (or any safety issue) during the open- |
| | label period of the study (and combined with |
| | the double-blind period). |
| To evaluate the effect of iptacopan on blood | Occurrence of clinically significant |
| pressures, heart rate, cardiac function and | changes from baseline in msDBP/msSBP (on- |
| biomarkers of cardiac injury during the 6- | site as well as home monitoring), heart rate, |
| month open-label period in adolescent | echocardiography parameters and NT- |
| participants. | proBNP |

### Study Design Rationale

The study design includes a 6-month double-blind, placebo-controlled period to evaluate the potential benefits of iptacopan in improving kidney function as compared to placebo. The duration of the placebo-controlled period is limited to 6 months because a longer period of placebo exposure and withholding of other experimental therapies such as immunosuppressive or anti-complement agents is considered unacceptable to both study participants and the investigators treating them (Bomback, A., Kavanagh D, Vivarelli M, et al (2022) Alternative Complement Pathway Inhibition With Iptacopan for the Treatment of C3 Glomerulopathy-Study Design of the APPEAR-C3G Trial. Kidney Int Rep; 7: 2150-9). Therefore, due to difficulty with recruiting ultra-rare disease patients into a longer study in this severe and progressive disease, prolonging the placebo-controlled trial beyond 6 months is considered not feasible.

This time period also enables us to balance the requirement of giving iptacopan for an adequate period of time to determine its effects on the kidney and to provide to all study participants the opportunity to receive a potentially disease-modifying therapy during the 6-month open-label iptacopan period that follows.

Enrollment is available to patients with biopsy-confirmed idiopathic IC-MPGN, aged 12 to 60 years. Adolescents are included in the study because their disease pathogenesis is similar to that of adults, including a central role of the overactivated AP as well as clinical characteristics of edema, severity of proteinuria, prevalence of nephrotic syndrome, hematuria and histopathology. Many patients are diagnosed in adolescence (median age at diagnosis of around 21 years —(Iatropoulos et al 2018) and given the fast-progressing nature of the disease there is a high unmet need for treatment in adolescents with IC-MPGN. The rationale for including participants with biopsy confirmed IC-MPGN and a substantial level of proteinuria is to enrich the population with participants who have both clear clinical manifestations of IC-MPGN and an increased risk for rapid disease progression. The purpose of the run-in period is to ensure that participants are receiving an ACEI/ARB at a stable maximally-tolerated dose and other immunosuppressive and antiproteinuric medications (MMF/MPS, corticosteroids and SGLT2i) at a stable dose for at least 90 days prior to randomization and the required vaccinations at least 2 weeks prior to the first dose of study drug. In addition, to minimize the potential carry-over treatment effects of prohibited medications (i.e., immunosuppressants other than mycophenolic acids or corticosteroids at doses greater than 7.5 mg/day), patients who take these treatments may have to discontinue the medications or reduce the dose to comply with the permitted and prohibited medication requirements of the study.

The primary endpoint for the study is the reduction in UPCR (sampled from a 24-hour urine collection) at 6 months of treatment with iptacopan compared to placebo. Key secondary endpoints include preservation of eGFR with iptacopan compared to placebo and a composite renal endpoint that requires: 1) stabilization of eGFR, and 2) a substantial reduction in proteinuria in order to designate a participant as having met the endpoint. The FACIT-Fatigue questionnaire will be used to assess iptacopan's effect on an important symptom reported by patients with IC-MPGN.

Additional analyses will be performed at the end of the open-label period comparing Day 360 to baseline (Day 1) proteinuria, eGFR, rate of the composite renal endpoint and FACIT-Fatigue score. These data are important for evaluating the durability of any effects seen at Day 180, as well as further improvements in these variables with a longer duration of treatment. Proteinuria reduction is a well-recognized surrogate endpoint reasonably likely to predict a treatment effect across a range of kidney diseases, e.g., slowing progression in several other renal diseases including diabetic nephropathy, FSGS (focal segmental glomerulosclerosis) and Immunoglobulin A (IgA) nephropathy (Thompson A, Carroll K, A Inker L, et al (2019) Proteinuria Reduction as a Surrogate End Point in Trials of IgA Nephropathy. Clin J Am Soc Nephrol; 14(3):469-81).

A key secondary endpoint for the study will evaluate the improvement in eGFR (measured as a continuous variable) afforded by iptacopan in comparison to placebo. In addition to the precise measurement of proteinuria reduction on a continuous scale (primary endpoint), a categorical definition of clinically important change in proteinuria (≥50% reduction in UPCR compared to the baseline) is included as a component of a composite renal secondary endpoint. A magnitude of 50% or greater reduction in UPCR would indicate potentially conversion from nephrotic range proteinuria (>3.5 g/day) to non-nephrotic range proteinuria, thereby decreasing edema, the risk of thrombosis, infection and other associated complications, while stabilizing eGFR in an IC-MPGN patient. In order to strengthen the clinical relevance of the definition, no more than a 15% decline in eGFR over the 6-month period is included as part of the composite renal endpoint. Selection of these endpoints in IC-MPGN presents the opportunity to determine if treatment with iptacopan provides improvements in renal function.

### Justification for Dose

The proposed iptacopan dose of 200 mg b.i.d. is considered appropriate based on data from the Phase 2 study, CLNP023X2202. Data for 16 native C3G patients demonstrated that C3 levels increased dose-dependently, whereas sC5b-9, Bb, the Wieslab assay and urine sC5b-9 decreased dose-dependently. Maximum decreases in plasma Bb, plasma sC5b-9 and urine sC5b-9 were obtained at 100 mg b.i.d.; however, maximum decreases in the Wieslab assay and increases in C3 were observed at 200 mg b.i.d. Dose-dependent, profound and sustained inhibition of alternative complement pathway was observed in both cohorts upon iptacopan treatment as demonstrated through: overall normalization of serum C3 levels, full inhibition of Wieslab activity at 200 mg b.i.d, reduction of plasma Bb, plasma and urine sC5b-9 (for Cohort A). In patients with native C3G (N=16), iptacopan at final dose levels of 200 mg b.i.d demonstrated a 45% reduction in UPCR from baseline to Week 12 (two-sided p=0.0003), as well as stabilization of renal function (SCR, CrCl, eGFR). In addition, chronic iptacopan monotherapy was well tolerated without unexpected or new safety findings. In native kidney C3G patients, no deaths or serious adverse events were observed during the treatment period. SAEs were observed in 1 patient during the run-in period (i.e., prior to any iptacopan treatment). There were no definitive discontinuations due to adverse events and no notable changes in hematology, biochemistry, urinalysis, vital signs or ECG parameters. Thus, the 200 mg b.i.d. dose of iptacopan appears to produce maximum changes in all of the complement biomarkers that have been evaluated to date in C3G patients.

### Rationale for Choice of Background Therapy

There are no licensed disease modifying therapies or “rescue therapies” with proven benefit available for the treatment of idiopathic IC-MPGN. KDIGO 2021 guidelines make recommendations for the treatment in idiopathic IC-MPGN. In line with KDIGO guidelines, all patients will be required to be on a maximum or maximally tolerated dose of an ACEI/ARB as supportive therapy for the duration of the trial (KDIGO 2021). Use of immunosuppressive therapy (mycophenolic acids, corticosteroids) is also allowed, as long as this is at a stable dose, also reflecting KDIGO guidelines (which recommend such treatments for more severe cases of IC-MPGN) and clinical practice, where such medications are used according to clinical judgement in some patients. Unless otherwise indicated, the practice guidelines are based upon very low-quality evidence, clinical experience, and expert opinion. Treatment is often influenced and determined by the severity of proteinuria and kidney dysfunction (KDIGO 2021).

The use of SGLT2 inhibitors is allowed; however, the dose must be stable during the Screening/run-in period and throughout the study drug treatment period. The use of corticosteroids and mycophenolic acid (mycophenolate mofetil (MMF) or mycophenolate sodium) is permitted during the course of the study as described in Table 3. All background therapy (ACEI/ARB, MMF/MPS, Corticosteroids and SGLT2i) will be considered as Auxiliary Medicinal Products (AMP) in this trial.

As a small molecule oral factor B (FB) inhibitor, iptacopan blocks FB and there by the AP, which plays an important role in the defense against microbes. Even though the other two complement initiating pathways (i.e., classic and lectin pathways) should not be attenuated by iptacopan, there is a theoretical risk that immunological responses to infection, especially against encapsulated bacteria, may be compromised in patients who take iptacopan. To adequately mitigate this risk, participants will be vaccinated against Neisseria meningitidis, Streptococcus pneumoniae and Haemophilus influenzae infections, based on local availability of vaccines, at least 2 weeks prior to the first dosing. In addition, the use of prophylactic antibiotics to prevent infection is permitted where this is standard of care therapy for managing IC-MPGN patients receiving anti-complement therapies at the site. Adverse events of infection will be closely monitored throughout the study. Participants will be required to carry Participant Cards that describe the symptoms of sepsis and meningitis for participant information and awareness and so they can inform their physician or the Investigator early in the onset of a potential infection. The card also informs medical professionals of the potential increased risk of bacterial infection and helps ensure timely diagnosis and treatment.

TABLE 3
Use of corticosteroids and mycophenolic acids

| Type of medication | Use of medication |
|---|---|
| Systemic | Participants are allowed to take prednisone at |
| corticosteroids | a dose ≤7.5 mg per day (or equivalent for |
| | similar medication) at any time during the |
| | course of the study. The dose of |
| | corticosteroids must be stable during the 90 |
| | day Screening/run-in period and throughout |
| | the study drug treatment period (change in |
| | dose is allowed if indicated due to |
| | Investigator concerns, an adverse event or |
| | other safety issue). |
| Topical corticosteroids | Allowed at any time. |
| Mycophenolic acids | Participants in all countries except India are |
| (MMF or | allowed to take mycophenolic acids at any |
| mycophenolate | time. The dose of mycophenolic acid must be |
| sodium) | stable during the 90 day Screening/run-in |
| | period and throughout the study drug |
| | treatment period (change in dose is allowed if |
| | indicated due to Investigator concerns, an |
| | adverse event or other safety issue). |
| | Participants in India are not permitted to take |
| | mycophenolic acids during the study as per |
| | the HA requirement. |

### Key Efficacy Estimands

Primary: proteinuria reduction quantified by the log-transformed ratio to baseline in UPCR
Secondary: a stable or improved eGFR compared to the baseline visit (≤15% reduction in eGFR) and ≥50% reduction in UPCR (based on 24-hour urine collection) compared to the baseline visit
Change from baseline in the Functional Assessment of Chronic Illness Therapy-Fatigue (FACIT-Fatigue) score

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating immune complex mediated membranoproliferative glomerulonephritis (IC-MPGN) in a subject in need thereof, the method comprising administering to the subject iptacopan or a pharmaceutically acceptable salt thereof to thereby treat the subject.

2. The method of claim 1, wherein the iptacopan or a pharmaceutically acceptable salt thereof is administered orally.

3. The method of claim 1 or 2, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, administered twice daily (b.i.d.), wherein the dosing amount refers to the anhydrous free base of iptacopan.

4. The method of claim 3, wherein the dose is about 200 mg, twice daily.

5. The method of any one of claims 1 to 4, wherein the pharmaceutically acceptable salt of iptacopan is iptacopan hydrochloride.

6. The method of any of claims 1 to 5, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.

7. The method of claim 6, wherein the ACEi or ARB has been administered at a maximally recommended or tolerated dose.

8. The method of claim 7, wherein the subject is further administered the supportive care at a stable dose.

9. The method of any of claims 1 to 8, wherein the subject is an adult or adolescent.

10. The method of any of claims 1 to 9, wherein a urine protein/creatinine ratio (UPCR) in the subject is reduced compared to prior to administering iptacopan or a pharmaceutically acceptable salt thereof.

11. The method of claim 10, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.

12. The method of any of claims 1 to 11, wherein an estimated glomerular filtration rate (eGFR) in the subject is stable or improved, compared to prior to administering iptacopan or a pharmaceutically acceptable salt thereof.

13. The method of claim 12, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.

14. Iptacopan or a pharmaceutically acceptable salt thereof for use in a treatment of immune complex mediated membranoproliferative glomerulonephritis (IC-MPGN) in a subject in need thereof, wherein the treatment comprises administering to the subject iptacopan or a pharmaceutically acceptable salt thereof.

15. The iptacopan or a pharmaceutically acceptable salt thereof for use of claim 14, wherein the iptacopan or a pharmaceutically acceptable salt thereof is administered orally.

16. The iptacopan or a pharmaceutically acceptable salt thereof for use of claim 14 or 15, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), wherein the dosing amount refers to the anhydrous free base of iptacopan.

17. The iptacopan or a pharmaceutically acceptable salt thereof for use of claim 16, wherein the dose is about 200 mg, twice daily.

18. The iptacopan or a pharmaceutically acceptable salt thereof for use of any one of claims 14 to 17, wherein the pharmaceutically acceptable salt of iptacopan is iptacopan hydrochloride.

19. The iptacopan or a pharmaceutically acceptable salt thereof for use of any of claims 14 to 18, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.

20. The iptacopan or a pharmaceutically acceptable salt thereof for use of claim 19, wherein the ACEi or ARB has been administered at a maximally recommended or tolerated dose.

21. The iptacopan or a pharmaceutically acceptable salt thereof for use of claim 20, wherein the subject is further administered the supportive care at a stable dose.

22. The iptacopan or a pharmaceutically acceptable salt thereof for use of any of claims 14 to 21, wherein the subject is an adult or adolescent.

23. The iptacopan or a pharmaceutically acceptable salt thereof for use of any of claims 14 to 22, wherein a urine protein/creatinine ratio (UPCR) in the subject is reduced compared to prior to administering iptacopan or a pharmaceutically acceptable salt thereof.

24. The iptacopan or a pharmaceutically acceptable salt thereof for use of claim 23, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.

25. The iptacopan or a pharmaceutically acceptable salt thereof for use of claims 14 to 24, wherein an estimated glomerular filtration rate (eGFR) in the subject is stable or improved, compared to prior to administering iptacopan or a pharmaceutically acceptable salt thereof.

26. The iptacopan or a pharmaceutically acceptable salt thereof for use of claim 25, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.

27. A pharmaceutical composition comprising a therapeutically effective amount of iptacopan or a pharmaceutically acceptable salt thereof for use in treating immune complex mediated membranoproliferative glomerulonephritis (IC-MPGN), in a subject in need thereof, wherein the pharmaceutical composition is to be administered to thereby treat the subject.

28. The pharmaceutical composition of claim 27, wherein the iptacopan or a pharmaceutically acceptable salt thereof is administered orally.

29. The pharmaceutical composition of claim 27 or 28, wherein the iptacopan or a pharmaceutically acceptable salt thereof is administered at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), wherein the dosing amount refers to the anhydrous free base of iptacopan.

30. The pharmaceutical composition of claim 29, wherein the dose is about 200 mg, twice daily.

31. The pharmaceutical composition of any one of claims 27 to 30, wherein the pharmaceutically acceptable salt of iptacopan is iptacopan hydrochloride.

32. The pharmaceutical composition of any one of claims 27 to 31, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.

33. The pharmaceutical composition of claim 32, wherein the ACEi or ARB has been administered at a maximally recommended or tolerated dose.

34. The pharmaceutical composition of claim 33, wherein the subject is further administered the supportive care at a stable dose.

35. The pharmaceutical composition of any one of claims 27 to 34, wherein the subject is an adult or adolescent.

36. The pharmaceutical composition of any one of claims 27 to 35, wherein a urine protein/creatinine ratio (UPCR) in the subject is reduced compared to prior to administering iptacopan or a pharmaceutically acceptable salt thereof.

37. The pharmaceutical composition of claim 35, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.

38. The pharmaceutical composition of any one of claims 27 to 37, wherein an estimated glomerular filtration rate (eGFR) in the subject is stable or improved, compared to prior to administering iptacopan or a pharmaceutically acceptable salt thereof.

39. The pharmaceutical composition of claim 38, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.

40. Use of iptacopan or a pharmaceutically acceptable salt thereof for the manufacture for treating immune complex mediated membranoproliferative glomerulonephritis (IC-MPGN), in a subject in need thereof.

41. The use of claim 40, wherein the iptacopan or a pharmaceutically acceptable salt thereof is administered orally.

42. The use of claim 40 to 41, wherein the use comprises administering to the subjects iptacopan or a pharmaceutically acceptable salt thereof at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), wherein the dosing amount refers to the anhydrous free base of iptacopan.

43. The use of claim 42, wherein the dose is about 200 mg, twice daily.

44. The use of any one of claims 40 to 43, wherein the pharmaceutically acceptable salt of iptacopan is iptacopan hydrochloride.

45. The use of any one of claims 40 to 44, wherein the subject has been administered a supportive care comprising a/an ACEi, ARB, mycophenolic acid (e.g., mycophenolate mofetil (MMF) and mycophenolate sodium (MPS)), corticosteroid, SGLT2 inhibitor, mineralocorticoid receptor antagonist, cyclophosphamide (CYC), or calcineurin inhibitor, prior to administering iptacopan or iptacopan or a pharmaceutically acceptable salt thereof.

46. The use of claim 45, wherein the ACEi or ARB has been administered at a maximally recommended or tolerated dose.

47. The use of claim 46, wherein the subject is further administered the supportive care at a stable dose.

48. The use of any one of claims 40 to 47, wherein the subject is an adult or adolescent.

49. The use of any one of claims 40 to 48, wherein a urine protein/creatinine ratio (UPCR) in the subject is reduced compared to prior to administering iptacopan or a pharmaceutically acceptable salt thereof.

50. The use of claim 49, wherein the urine protein/creatinine ratio (UPCR) in the subject is reduced by no less than 50% compared to prior to administering iptacopan or a pharmaceutically acceptable salt thereof, in particular wherein the urine protein/creatinine ratio (UPCR) in the subject is reduced by no less than 50% after 6 months of treatment.

51. The use of any one of claims 40 to 50, wherein an estimated glomerular filtration rate (eGFR) in the subject is stable or improved, compared to prior to administering iptacopan or a pharmaceutically acceptable salt thereof.

52. The use of claim 51, wherein the estimated glomerular filtration rate (eGFR) in the subject is reduced by no greater than 15%, compared to prior to administering iptacopan or a pharmaceutically acceptable salt thereof, in particular wherein the estimated glomerular filtration rate (eGFR) in the subject is reduced by no greater than 15% after 6 months of treatment.
